(19) United States
(12) Patent Application Publication
(10) Pub. No.: US 2009/0163684 A1
(43) Pub. Date: Jun. 25, 2009

(54) METHOD FOR PURIFYING POLAR VINYL COMPOUNDS

(75) Inventors: Bernd Judat, Mannheim (DE); Matthias Rauls, Ludwigshafen (DE); Martin Rübenacker, Altrip (DE); Manfred Winter, Dittelsheim-Hessloch (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 11/720,659

(22) PCT Filed: Nov. 29, 2005
(86) PCT No.: PCT/EP05/12733

§ 371 Date: Jun. 1, 2007

(30) Foreign Application Priority Data
Dec. 1, 2004 (DE) 10 2004 058 071.5

(51) Int. Cl. C08F 120/54; C07C 233/01

(52) U.S. Cl. 526/303.1; 564/192

(57) ABSTRACT

Method of purifying an open-chain N-vinyl compound by crystallization in a crystallizer, the crystallization being carried out from a melt of a mixture comprising open-chain N-vinyl compound at a pressure of 10⁻³ to 400 bar.

The present invention relates to a method of purifying polar vinyl compounds, and in particular to the crystallization of open-chain N-vinyl compounds.

Polar vinyl compounds for the purposes of the present invention are open-chain mono-ethylenically unsaturated monomers further comprising nitrogen as heteroatom.

From such vinyl compounds homopolymers and copolymers are prepared by means of polymerization and find application in a wide variety of sectors, such as in the cosmetics and drug industries, for example, and also in the paper industry.

Vinyl compounds, because of the double bond, are highly reactive and tend readily toward uncontrolled polymerization. Consequently, in order for improved handling during storage and transit, for example, polymerization inhibitors, which are intended to prevent uncontrolled polymerization, are added to the vinyl compounds. A disadvantage of this is that without a further purification step it is not possible to prepare high molecular mass homopolymers and copolymers from the vinyl compounds comprising polymerization inhibitors, since the polymerization inhibitors control the polymerization and the molecular weight of the polymers is thereby limited.

High molecular mass polymers of this kind, however, comprising no impurities such as polymerization inhibitors, are desirable for many areas of application.

For preparing high molecular mass polymers, therefore, high-purity vinyl monomers are required, but are difficult to obtain on account of the polymerization tendency described above.

EP 1 048 646 A1 describes a process for continuous distillation of thermolabile monomers such as N-vinyl compounds under reduced pressure in the presence of form amide. The product obtainable by that process still has a formamide fraction of less than 5% by weight, and so its polymerization to a high molecular mass polymer is not possible.

U.S. Pat. No. 6,033,530 discloses a method of purifying thermolabile monomers such as N-vinyl-formamide by means of a heterogeneous azeotropic distillation in the presence of a distillation auxiliary.

Another way of preparing high-purity vinyl compounds is to remove the impurity via ion exchange resins or activated carbon. The regeneration of these components in the columns packed with them must, however, be carried out at certain intervals of time, which makes industrial application more difficult.

Japanese laid-open specification JP-A 61-286069 describes an extractive separation process in which water and aromatic hydrocarbon solvents are used. A disadvantage of this process is that some vinyl compounds, such as N-vinylcarboxamides, for example, are unstable in water and tend toward hydrolysis.

EP 0 644 180 A1 discloses a process for preparing high-purity polar vinyl compounds in which a crystallization is carried out under high pressures (500-3000 atm) and temperatures (0-100° C.). The crystallization is carried out in two steps: in a first step, the polar vinyl component is crystallized under pressure. The crystals are separated from the liquid phase that remains. This liquid phase is enriched with contaminants and in a second step is crystallized again. The second crystallizate is mixed into the crude vinyl compound, which in turn is passed to the first crystallization. A disadvantage of this process are the high operating costs and capital costs, owing to the high pressures.

German laid-open specification DE 195 36 792 A1 describes a process for separating material from a liquid mixture by crystallization, in which a two-phase seed layer in the form of a melt or solution of the composition to be separated, with crystals already suspended therein, is applied to those surfaces from which it is intended that crystals should grow in the course of the crystallization. The process pertains generally to liquid mixtures suitable for separation, with a melting point between −50° C. to +300° C., suitability being possessed in particular by compounds including N-vinylpyrrolidone, naphthalene and acrylic acid.

DE 195 36 859 A1 discloses a method of purifying N-vinylpyrrolidone by crystallization in which the surfaces of the crystallizer from which it is intended that the crystals should grow are covered with a seed layer of N-vinylpyrrolidone.

A disadvantage of the process and method described in German laid-open specifications DE 195 36 792 A1 and DE 195 36 859 A1, respectively, is the inconvenience of covering the crystallizer surfaces with a seed layer.

In numerous fields of application there is a great interest in high-purity open-chain N-vinyl compounds, particularly N-vinylformamide, which comprise no impurities such as polymerization inhibitors and from which high molecular mass homopolymers and copolymers can be prepared.

The present invention was based on the object of finding a method of purifying an open-chain N-vinyl compound that avoids the disadvantages of the prior-art processes.

This object has been achieved by means of a method of purifying an open-chain N-vinyl compound by crystallization in a crystallizer, crystallization taking place from a melt of a mixture comprising open-chain N-vinyl compound at a pressure of 10⁻³ to 400 bar.

Advantages in comparison to the prior-art processes include the facts that the method of the invention operates without the use of solvents and can be conducted under moderate pressures and with economic energy consumption.

By open-chain N-vinyl compounds for the purposes of the present invention are meant open-chain monoethylenically unsaturated vinyl compounds further comprising nitrogen as heteroatom. The position of the nitrogen relative to the double bond is unimportant. The method of the invention can be practised either as a layer crystallization or as a suspension crystallization.

The pressure during crystallization in accordance with the method of the invention is between 10⁻³ to 400 bar, preferably between 10⁻² and 250 bar, more preferably between 10⁻¹ and 100 bar and in particular between 10⁻¹ and 50 bar. Particular advantage attaches to conducting the method of the invention at atmospheric pressure. The pressure figures indicated should not be regarded as being absolute, with fluctuations in the region of ±250 mbar being naturally possible.

The temperature within the crystallizing melt is in the range from 0.1 to 40 K below the melting point of the pure melt, preferably in the range from 0.2 to 20 K and more preferably in the range from 0.5 to 10 K below the melting point of the pure melt.

The method of the invention starts from a mixture which comprises open-chain N-vinyl compound and is to be purified by crystallization, this mixture also being referred to below as crude N-vinyl compound. Besides the open-chain N-vinyl compound the crude N-vinyl compound comprises polymerization inhibitors and secondary components which come, for example, from the synthesis of the open-chain N-vinyl compound. These compounds are referred to collectively below as impurities.

Typical secondary components of such kind are aldehydes such as, for example, acetaldehyde, formaldehyde and crotonaldehyde, but also other secondary components are possible, such as reactants, auxiliaries and solvents from the preparation of the open-chain N-vinyl compound.

Generally speaking, polymerization inhibitors comprised in the crude N-vinyl compound are N-oxyls (nitroxyl radicals or N-oxyl radicals, compounds containing at least one >N—O. group), examples being 4-hydroxy-2,2,6,6-tetramethylpiperidine-N-oxyl, 4-oxo-2,2,6,6-tetramethylpiperidine-N-oxyl, 4-methoxy-2,2,6,6-tetramethylpiperidine-N-oxyl and 2,2,6,6-tetramethylpiperidine-N-oxyl. It will be appreciated that the crude N-vinyl compound may also comprise other polymerization inhibitors which can be used for stabilizing ethylenically unsaturated compounds. Suitable stabilizers are, generally, phenolic compounds, said N-oxyls, aromatic amines, phenylenediamines, imines, sulfonamides, oximes, oxime ethers, hydroxylamines, urea derivatives, phosphorus compounds, sulfur compounds such as phenothiazine, complexing agents and metal salts, and mixtures thereof.

The crude N-vinyl compound may originate from any preparation process of the open-chain N-vinyl compound. As the crude N-vinyl compound it is preferred to use product streams which come already from a distillative purification. Such product streams are commonly taken from the side offtake or the top of the distillation column. The distillative purification of N-vinyl compounds is described for example in the aforementioned specifications EP 1 048 646 A1 and U.S. Pat. No. 6,033,530 and also EP 0 231 901 A1.

A particularly preferred product stream used as crude N-vinyl compound is that from distillative purification, which on account of its high impurities fraction is unsuitable for polymerization. This product stream generally comprises less than 40%, preferably less than 20%, more preferably less than 10% and very preferably less than 5% by weight of impurities, based on the crude-N-vinyl compound; that is, the amount of open-chain N-vinyl compound is generally at least 60%, preferably at least 80%, more preferably at least 90% and very preferably at least 95% by weight, based on the crude N-vinyl compound.

The open-chain N-vinyl compound is crystallized one or more times, preferably once or twice, until the desired purity is reached. In this context it is preferred to operate in accordance with the countercurrent principle: in other words, the mother liquor from the respective crystallization stage is supplied to the respective preceding crystallization stage. If appropriate, further purification steps are carried out.

In the respective crystallization stage the crystallization is preferably taken to a point where at least 5%, preferably at least 10% and more preferably at least 20% by weight of the open-chain N-vinyl compound is crystallized out. Typically, in one crystallization stage, not more than 90%, preferably not more than 80% and in particular not more than 70% by weight of the open-chain N-vinyl compound used in the respective crystallization stage is crystallized out in order to achieve an adequate purification effect.

The crystallizer which can be used in the method of the invention is not subject per se to any restriction. Crystallizers which have proven particularly suitable are those whose function is based on the formation of crystals on cooled surfaces. Crystallization techniques of this kind are also referred to as layer crystallization. Suitable apparatus is found in the patent specifications indicated in DE 102 57 449 A1 on page 4 lines 6 and 7.

In one embodiment of the method of the invention the open-chain N-vinyl compound is crystallized with cooling. In this form of layer crystallization the crystals are separated from the mother liquor and melted.

For the layer crystallization the crude N-vinyl compound for purification is brought into contact with a cooling surface, examples being the cooled surfaces of a heat exchanger. The heat exchanger surfaces of the crystallizer are cooled preferably to temperatures up to 40° C. below the melting temperature of the open-chain N-vinyl compound. When the desired degree of crystallization is reached the cooling operation is ended and the remaining liquid (mother liquor) is taken off, by pumping or under gravity flow, for example. The purity of the open-chain N-vinyl compound crystals which remain on the heat exchanger surfaces of the crystallizer can be raised further by liquefying more highly contaminated fractions of the crystals by means of partial melting (sweating) and taking off this liquid. Another possibility is to raise the purity of the crystals on the heat exchanger surfaces by washing with a washing liquid. Examples of suitable washing liquids include the liquid pure product, i.e., the open-chain N-vinyl compound with the desired final purity, which is obtained by melting the crystals, or the liquid crude N-vinyl compound. It should be ensured, however, that the washing liquid has a higher purity than the mother liquor from which the crystallizate has been separated. Washing or sweating is described in more detail later on below and under certain circumstances may make a further crystallization stage unnecessary.

The purified, crystallized, open-chain N-vinyl compound is isolated customarily by melting the crystallized open-chain N-vinyl compound, by for example heating the heat exchanger surfaces to a temperature above the melting temperature of the open-chain N-vinyl compound and/or by supplying for example a melt of purified open-chain N-vinyl compound. In these cases the purified open-chain N-vinyl compound is produced as a melt and is isolated as such. The crystalline open-chain N-vinyl compound can also be dissolved in water or an appropriate solvent and the resulting solution used directly in the subsequent polymerization.

The temperature required for the layer crystallization depends on the degree of impurity. The upper limit is of course the temperature at which the already crystallized openchain N-vinyl compound is in equilibrium with the open-chain N-vinyl compound comprised in the mother liquor (equilibrium temperature). Depending on the composition of the crude N-vinyl compound the equilibrium temperature is situated in the range from 0.1 to 40 K below the equilibrium temperature of the pure N-vinyl compound. Preferably the equilibrium temperature of the crude N-vinyl compound is in the range from 0.2 to K and more preferably in the range from 0.5 to 10 K below the equilibrium temperature of the pure N-vinyl compound.

In one embodiment of the crystallization method the layer crystallization is conducted in the presence of seed crystals.

Crystallization on cooling surfaces can be conducted as a dynamic or static technique. Dynamic techniques are known for example from EP 0 616 998 A1, static ones from U.S. Pat. No. 3,597,164, for example. In the case of the dynamic crystallization techniques the crude product for crystallization is held in a flowing motion. This can be done by means of a forced flow in fully flow-traversed heat exchangers, as described in DE 26 06 364 A1, or by means of a trickle film onto a cooled wall, such as cooling rolls or cooling belts. In the case of static crystallization, mass transfer takes place in the liquid phase only by means of free convection (resting melt). Layer crystallization on cooling surfaces in dynamic operation of the technique is preferred in the present invention.

Static layer crystallization is preferably initiated with a seed procedure. In one particular embodiment of the seed procedure the liquid which remains as a residual film on the cooling surfaces after melting is partly or fully frozen on the cooling surface, as seed crystallizate, and subsequently a further crystallization is carried out. Seed crystallizate can also be frozen by applying seed crystallizate to the cooling surface prior to crystallization by contacting the cooling surface in a separate step with a melt of the crude N-vinyl compound that is of greater purity, relative to the liquid composition to be separated, subsequently separating the one from the other, and then forming a corresponding seed crystallizate by cooling. In this case as well the residual film which remains on the cooling surfaces is partially or fully frozen by lowering the temperature on the surfaces. Additionally, for producing a seed crystal layer, the cooling surface can be contacted with a crystal-containing suspension of the crude N-vinyl compound, in order to obtain a seed crystal layer on the cooling surface by cooling thereof after the suspension has been removed. Seeding can also be achieved by adding crystals in solid or suspension form to the melt of the crude N-vinyl compound, with the melt in this case being at a temperature close to or below the dissolution temperature. Seeding can also be achieved by generating and/or maintaining a crystal layer on a locally limited, separately cooled cooling surface (known as a cold spot). Alternatively cooling can also be carried out directly by adding a coolant (e.g., dry ice).

Crystallization on cooling surfaces is preferably carried out in one stage; that is, the required final purity of the open-chain N-vinyl compound is achieved after just one crystallization stage. The purity can be raised further by carrying out the crystallization in a plurality of stages, in the form of what is known as fractional crystallization. By repeated crystallization of the pure fractions that are formed in each case it is possible to adjust the desired final purity of the open-chain N-vinyl compound.

Fractional crystallization can also be employed in respect of other suitable crystallization techniques, such as that of suspension crystallization, for instance.

Suspension crystallization can be carried out as an alternative to layer crystallization. In the case of suspension crystallization a crystal suspension in a melt enriched in impurities is produced by cooling the crude product, thus in this case the crude N-vinyl compound. The crystals are distributed dispersely in the liquid phase (mother liquor) and may grow directly in the suspension (melt) or may deposit as a layer on a cooled wall. Subsequently, on reaching a desired crystal content, normally 5% to 40% by weight, the crystals are scraped from said wall and suspended in the residual melt. The crystal suspension is preferably agitated during the process, in particular by being pumped in circulation or stirred. This is necessary because of the high densities of solids in the case of suspension crystallization and because of the large temperature gradients, which can lead to incrustation of the heat transfer surfaces. Besides the stirred tanks that are usual in solution crystallization, other apparatus as well is employed, such as the scraped-surface cooler, for example. The crystal layer which forms is generated within a jacketed tube, which is flow-traversed internally and cooled from the outside, and is taken off by slow-rotating scraper elements and conveyed back into the melt. The crystals may subsequently pass through a growth zone, in which they are able to continue growing in the case of supersaturation. Another apparatus frequently used is the cooling disk crystallizer. In this case the crystals are formed on cooled disks which dip into the melt and are wiped off continuously by means of scrapers. Besides these suspension crystallization techniques with indirect cooling via heat exchange elements, the suspension can also be cooled directly by the introduction of a coolant (e.g., cold gases or liquids, or evaporating liquids).

Suspension crystallization is preferably initiated with a seeding operation. Seeding can be brought about by adding crystals in solid form or in suspension form to the melt of the crude N-vinyl compound, the melt then being, at the time of addition, at a temperature close to or below the dissolution temperature. The crystals added may be specially treated, e.g., size-reduced and/or washed. Seeding can also be brought about by producing and/or maintaining a crystal layer on a locally limited, separately cooled cooling surface (known as a cold spot). Seed crystals can also be removed from a separately cooled surface of this kind (mechanically, for example, or by flow forces or by ultrasound) and carried into the melt of the crude N-vinyl compound. Alternatively, cooling can also be carried out directly by adding a coolant (e.g., dry ice).

Seeded operation of the crystallization can also be accomplished by first sharply cooling the liquid melt, until crystal formation begins, spontaneously or with application of an above-described seeding operation, then raising the temperature of the suspension again, in order to melt a large fraction of the resultant crystallizate, and then carrying out cooling more slowly, with control, in the presence of the remaining residual crystallizate (seed crystals), in order to produce the desired suspension.

Suspension crystallization can be operated continuously or batchwise, preferably continuously.

Suitable methods for separating the liquid phase (mother liquor) from an open-chain N-vinyl compound crystallized by suspension crystallization include all known methods of solid/liquid separation, by means for example of a centrifuge or filtration. Centrifuging or filtering may be preceded by thickening of the suspension, by means of hydrocycones, for example. Filtration may take place discontinuously or continuously, under superatmospheric or reduced pressure. When suction filters are used, they may have a stirrer mechanism.

During and/or after the solid/liquid separation there may be further process steps, examples being washing and sweating, for the purpose of increasing the purity of the crystals and/or of the crystal cake. In the case of washing, the amount of washing liquid is preferably between 5 and 500 g, more preferably between 10 and 300 g, very preferably between 15 and 50 g of washing liquid per 100 g of crystallizate. Examples of suitable washing liquids include the liquid pure product, in other words the open-chain N-vinyl compound with its desired final purity, as obtained by melting of the crystals, or the liquid crude N-vinyl compound. It must be ensured, however, that the washing liquid has a higher purity than the mother liquor from which the crystallizate has been separated. In certain circumstances, washing or sweating may make a further crystallization stage unnecessary.

Washing can be carried out in apparatus suitable for this purpose. It is advantageous to use washing columns, in which the separation of the mother liquor and the washing take place in one step; centrifuges operated in one or more stages; and also suction filters or belt filters. On both centrifuges and belt filters the washing can be carried out in one or more stages. If the crystallization itself is operated in a static crystallizer, then washing is advantageously conducted in the crystallizer itself.

Sweating comprises a local melting of impure regions of the crystals. For this purpose the temperature of the crystal layer is raised slightly, by 0.5 to 5° C. above the melting temperature, for example, and the regions of the crystal layer that have a greater level of impurities melt, thereby producing an additional purification effect. The sweated product is then supplied to the mother liquor and processed further together with it. The amount of sweat material is advantageously between 1 and 35 g, preferably between 10 and 30 g of melted crystallizate per 100 g of crystallizate prior to sweating. If the crystallization itself is operated in a static crystallizer, then sweating is advantageously conducted in the crystallizer itself.

Implementing a combination of washing and sweating in one apparatus is also suitable for increasing the purity of the crystals and/or of the crystal cake.

The open-chain N-vinyl compound produced by crystallization has a purity of ≧98%, preferably ≧99%, more preferably ≧99.5% and in particular ≧99.9%.

The present invention provides a method of purifying open-chain monoethylenically unsaturated vinyl compounds which additionally comprise nitrogen as heteroatom. The position of the nitrogen relative to the double bond is unimportant. These N-vinyl compounds include, for example, N-vinylcarboxamides.

Generally speaking, the open-chain N-vinyl compounds can be described with the aid for example of the following formula:

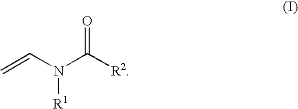

In this formula, R¹ and R² can be identical or different and can be hydrogen and C₁ to C₆ alkyl. Monomers of this kind are, for example, N-vinylformamide (R¹═R²═H in the formula (I)), N-vinyl-N-methylformamide, N-vinylacetamide, N-vinyl-N-methylacetamide, N-vinyl-N-ethylacetamide, N-vinyl-N-methylpropionamide and N-vinylpropionamide. The method of the invention is especially suitable for preparing high-purity N-vinyl-formamide.

Monomers which can likewise be purified in accordance with the invention include those of the formula (II):

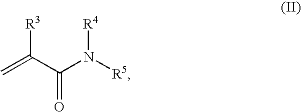

in which R³, R⁴ and R⁵ are identical or different. R³ can be hydrogen or C₁ to C₆ alkyl, R⁴ and R⁵ independently of one another can be hydrogen or a C₁ to C₆ alkyl, preferably C₂-C₄ alkyl, which is optionally substituted by a hydroxyl group, a dialkylamino group, a sulfate group or a quaternary ammonium group. Examples of monomers of this kind are acrylamides such as N-methylacrylamide, N-ethylacrylamide, N-isopropyl-acrylamide, monomethylolacrylamide, diacetoneacrylamide, N,N-dimethylacrylamide, N,N-diethylacrylamide, N,N-methylenebisacrylamide, 2-acrylamido-2-methylpropane-sulfonic acid or its sodium salt and N-methylolacrylamide and also, of the stated compounds, the methacrylamide derivatives.

The method of the invention is used to particular advantage to purify N-vinylformamide. The advantage over known methods is in particular that monomer qualities are obtained which can be processed to particularly high molecular mass polymers. Thus, for example, from N-vinylformamide crystallized in accordance with the invention, by the process of oil-in-water emulsion polymerization, poly-N-vinylformamides are obtained which have K values according to Fikentscher of more than 230 (measured in 5% strength by weight aqueous sodium chloride solution at 25° C., at a pH of 7 and at a polymer concentration of 0.1% by weight). The preparation of poly-N-vinylformamides with such high molecular masses is difficult because even impurities of the order of a few ppm considerably influence the polymerization of N-vinylformamide.

The present application therefore likewise provides for the preparation of high molecular mass homopolymers and copolymers from the open-chain N-vinyl compounds, particularly of poly-N-vinylformamide, the K values being preferably above 230.

Further provided by the present application is the use of the high molecular mass homopolymers and copolymers in the paper, drug or cosmetics industry. The purpose of the example which follows is to illustrate the invention, though without restricting it.

The K value was determined by the method described above according to H. Fikentscher, Cellulose-Chemie, Volume 13, 58-64 and 71-74 (1932) (measured in 5% strength by weight aqueous sodium chloride solution at 25° C., a pH of 7 and a polymer concentration of 0.1% by weight).

The percentages in the example are by weight unless indicated otherwise.

In the subsequent polymerization of high-purity N-vinylformamide to high molecular mass polyvinylformamide, the following emulsifiers were used:

Span® 80: sorbitan monooleate from ICI

Hypermer® B246: polyester-polyethylene oxide-polyester block copolymer having a molar mass >1000 g/mol, prepared by reacting condensed 12-hydroxystearic acid with polyethylene oxide in accordance with the teaching of EP 0 000 424.

### EXAMPLES

### Example 1

### Preparation of High-Purity N-Vinylformamide (Static Layer Crystallization)

3070 g of a melt of N-vinylformamide having a purity of about 97.5% by weight with impurities comprising formamide, crotonaldehyde and further impurities were introduced under atmospheric pressure into a vertical 3-liter jacketed tube having a diameter of 50 mm and were cooled to −11° C. and induced to crystallize by addition of a small amount of dry ice. By heating to −9.5° C. a large part of the crystallizate formed was dissolved again, so that only a few seed crystals remained in the melt. Thereafter, cooling took place at a rate of 0.3 K/h to a temperature of −12.5° C. in 10 hours, until about 1880 g had frozen out. At this temperature the residual melt was run off into a vessel. The crystallizate was subsequently partially remelted (sweating) with a heating rate of 0.5 K/h to a temperature of −8° C. The melted mass was likewise run off from the crude crystallizer into a vessel, leaving a mass of 1490 g of crystallizate in the crystallizer. In order to remove this purified product from the crystallizer the temperature was increased further and the crystallizate was melted completely again and run off into a separate vessel. The purity of the crystallizate obtained by melting was found to be >99.5% by weight.

Polymerization of high-purity N-vinylformamide to high molecular mass poly-N-vinylformamide

A polymerization reactor with a capacity of 2 l, equipped with anchor stirrer, reflux condenser, thermometer and nitrogen inlet, is charged, with stirring, with the following substances: 256.1 g of a hydrocarbon mixture with a boiling range of 192 to 254° C. (Shell-solo D70), 9 g of Span® 80 and 3 g of Hypermer® B246. Added to this initial charge is a solution of 5.88 g of 75% strength phosphoric acid, 7.92 g of 25% strength sodium hydroxide solution and 303 g of the high-purity freshly crystallized N-vinylformamide in 383 g of water with a pH of 6.5. The contents of the vessel are emulsified for 1 hour with a stirring speed of 350 rpm and with introduction of 10 l/h nitrogen. Subsequently, at a stirring speed of 250 rpm, 0.45 g of 2,2′-azobis(4-methoxy-2,4-dimethylvaleronitrile) and 0.15 g of 2,2′-azobis(2,4-dimethylvaleronitrile), in suspension in 10 g of hydrocarbon mixture (Shellsol® D70), are added over a period of 6 hours. Stirring was carried out at 30-31° C. for a total of 15 hours, followed by polymerization to completion at 40° C. for 4 hours more.

The K value according to Fikentscher was 235. It was no longer possible to detect crotonaldehyde. The fraction of formamide had been reduced to approximately one third.

### Example 2

### Preparation of High-Purity N-Vinylformamide (Suspension Crystallization)

1800 g of a crude solution of N-vinylformamide with a 0.69% formamide impurity and with further impurities in the ppm range were introduced under atmospheric pressure into a vertical 1.5 liter tubular crystallizer equipped with a close-clearance helical stirrer, and were cooled from −8.3° C. to −10.3° C. at 0.5 K/h. In the course of cooling, crystals were formed in the melt, and were held in suspension by the stirring element. When the final temperature was reached, the proportion of solids in the crystallizer was approximately 42% by weight. The contents of the crystallizer were separated off on a screen bowl centrifuge at 2000 min⁻¹ over the course of 3 minutes. One portion of the crystallizate was analyzed using a gas chromatograph. 0.08% formamide was found.

Polymerization of high-purity N-vinylformamide to high molecular mass poly-N-vinylformamide

Another portion of the crystallizate was polymerized to high molecular mass poly-N-vinylformamide as described in example 1. The K value according to Fikentscher was 228.

1. A method of purifying an open-chain N-vinyl compound by crystallization in a crystallizer, which comprises crystallizing from a melt of a mixture comprising open-chain N-vinyl compound at a pressure of 10⁻³ to 400 bar.

2. The method according to claim 1, wherein crystallization is conducted at a pressure of from 10⁻¹ to 50 bar.

3. The method according to claim 1, wherein crystallization is conducted at atmospheric pressure.

4. The method according to claim 1, comprising layer crystallization.

5. The method according to claim 1, comprising suspension crystallization.

6. The method according to claim 1, wherein crystallization is conducted as fractional crystallization.

7. The method according to claim 1, wherein the crystal purity is raised by washing and/or sweating.

8. The method according to claim 1, wherein the mixture comprising open-chain N-vinyl compound is a crude N-vinyl compound which besides the open-chain N-vinyl compound comprises polymerization inhibitors and secondary components.

9. The method according to claim 8, wherein the amount of open-chain N-vinyl compound in the crude N-vinyl compound is at least 90% by weight.

10. The method according to claim 1, wherein the open-chain N-vinyl compound is selected from the compounds of the general formula I 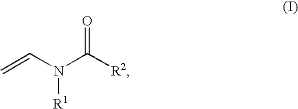
in which R¹ and R² can be identical or different and are hydrogen and C₁ to C₆ alkyl and compounds of the general formula II 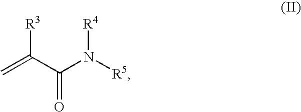
in which R³, R⁴ and R⁵ can be identical or different and R³ is hydrogen or C₁ to C₆ alkyl and R⁴ and R⁵ independently of one another can be hydrogen or a C₁ to C₆ alkyl group which is optionally substituted by a hydroxyl group, a dialkylamino group, a sulfate group or a quaternary ammonium group.

11. The method according to claim 10, wherein the compounds of the general formula I comprise N-vinylformamide, N-vinyl-N-methylformamide, N-vinyl-acetamide, N-vinyl-N-methylacetamide, N-vinyl-N-ethylacetamide, N-vinyl-N-methylpropionamide and N-vinylpropionamide.

12. The method according to claim 10, wherein the compounds of the general formula II comprise acrylamides such as N-methylacrylamide, N-ethylacrylamide, N-isopropylacrylamide, monomethylolacrylamide, diacetoneacrylamide, N,N-dimethylacrylamide, N,N-diethylacrylamide, N,N-methylenebisacrylamide, 2-acrylamido-2-methylpropanesulfonic acid or its sodium salt and N-methylolacrylamide and also, of the compounds stated, the methacrylamide derivatives.

13. The method according to claim 11, wherein the open-chain N-vinyl compound is N-vinylformamide.

14. A process for preparing high molecular mass homopolymers and copolymers, which comprises synthesizing them from the open-chain N-vinyl compounds prepared according to claim 1.

15. The process according to claim 14, wherein the high molecular mass homopolymers and copolymers comprise poly-N-vinylformamide having a K value of more than 230.

16. A high molecular mass polymer for use in the paper drug or cosmetics industry wherein the high molecular mass polymer is prepared by the process according to claim 14.

17. A paper comprising a high molecular mass polymer prepared by the process according to claim 14.

18. A drug comprising a high molecular mass polymer prepared by the process according to claim 14.

19. A cosmetic comprising a high molecular mass polymer prepared by the process according to claim 14.
